(19) United States
(12) Patent Application Publication
(10) Pub. No.: US 2008/0183230 A1
(43) Pub. Date: Jul. 31, 2008

(54) Methods and Apparatus for Selectively Shunting Energy in an Implantable Extra-Cardiac Defibrillation Device

(76) Inventors: Gary Kemmetmueller, Rogers, MN (US); W. William Wold, Edina, MN (US)

Correspondence Address: MEDTRONIC, INC., 710 MEDTRONIC PARKWAY NE, MINNEAPOLIS, MN, 55432-9924, US

(21) Appl. No.: 11/554,873

(22) Filed: Jan. 26, 2007

(51) Int. Cl. A61N 1/05

(52) U.S. Cl. 607/5

(57) ABSTRACT

The disclosure provides methods and apparatus for simultaneously providing protection to an implantable medical device, such as an extra-cardiac implantable defibrillator (EID), while allowing efficacious therapy delivery via an external defibrillator (e.g., an automated external defibrillator, or AED). Due to the orientation of the electrodes upon application of therapy via, for example, via an AED the structure of the EID essentially blocks therapy delivery. In addition, but for the teaching of this disclosure sensitive circuitry of an EID can be damaged during application of external high voltage therapy thus rendering the EID inoperable. EIDs are disclosed that are entirely implantable subcutaneously with minimal surgical intrusion into the body of the patient and provide distributed cardioversion-defibrillation sense and stimulation electrodes for delivery of cardioversion-defibrillation shock and pacing therapies across the heart when necessary. Configurations include one hermetically sealed housing with one or, optionally, two subcutaneous sensing and cardioversion-defibrillation therapy delivery leads or alternatively, two hermetically sealed housings interconnected by a power/signal cable. The housings are generally dynamically configurable to adjust to varying rib structure and associated articulation of the thoracic cavity and muscles. Further the housings may optionally be flexibly adjusted for ease of implant and patient comfort. One aspect includes partially insulating a surface of an EID that faces away from a heart while maintaining a major conductive surface facing the heart.

### FIELD OF THE INVENTION

The present invention relates to the field of chronically implantable medical devices; in particular, the invention relates to methods and apparatus to selectively shunt externally-delivered defibrillation energy delivered to a subject who has an extra-cardiac implantable defibrillator (EID) to preserve the EID and to allow the externally-delivered defibrillation waveform and its accompanying therapeutic energy to reach the myocardium.

### BACKGROUND OF THE INVENTION

Both automated external defibrillators (AEDs) and implantable cardioverter-defibrillators (ICDs) are becoming increasing available and it is estimated that as a result many thousands of individuals have received life-saving defibrillation therapy.

More recently non-transvenous, extra-cardiac ICDs—herein EIDs (whether or not such devices include cardioversion capability)—have begun to be developed and might become as widespread as ICDs are today. As a result, the possibility exists that an individual having an EID might receive high energy defibrillation therapy from an AED.

The inventors suggest that for a number of reasons such therapy could cause more harm than good unless preventative measures are incorporated into the EID.

Prior U.S. Pat. No. 5,999,398 to Makl et al. issued 7 Dec. 1999 (the '398 patent) entitled, “Feed-through Assembly having Varistor and Capacitor Structure,” is hereby incorporated herein by reference. In the '398 patent a filter structure is proposed that includes both varistor and capacitive characteristic thereby providing purportedly effective transient suppression and interference filtering with a single package. Although not central to the present invention, prior U.S. Pat. No. 6,253,105 to Leyde entitled, “Method for Delivering Defibrillation Energy,” is also incorporated herein by reference in its entirety.

### SUMMARY

The present invention provides methods and apparatus for simultaneously providing protection to an implantable medical device, such as an extra-cardiac implantable defibrillator (EID), and allowing efficacious therapy delivery via an external defibrillator (e.g., a manual or an automated external defibrillator, or AED). Due to the orientation of the electrodes upon application of therapy via, for example, an AED the structure of the EID can essentially block therapy delivery. In addition, sensitive circuitry of an EID can be damaged thus rendering the EID inoperable.

In one form of the invention, an EID includes a pair of high voltage-capacity defibrillation electrodes defining at least one defibrillation vector through a volume of myocardial tissue and at least one voltage shunting device (e.g., a varistor such as a metal oxide varistor, or MOV). As is known in the electronic arts a varistor is a voltage dependent, nonlinear device that has electrical characteristics similar to a pair of Zener diodes mounted back-to-back. Basically, a varistor shunts transient electrical currents away from circuitry by presenting a low resistance path in the presence of overvoltage situations. They are the most broadly applied technology, protecting vulnerable circuit components in applications whether low or high energy and current ratings are required. Commercially available varistors are available with operating voltages from 2.5V to 2800VDC and 3.5-3500VDC from companies such as Littelfuse, Inc. of Des Plaines, Ill. The Littelfuse company sells MOVs composed mainly of zinc oxide with small amounts of bismuth, manganese, cobalt, and other metal oxides that work by absorbing voltage surges and dissipating the energy as heat. These MOVs are available with peak current ratings ranging from 40 A to 70,000 A and peak energy ratings ranging from 0.1 J to 10,000 J. Certain Littelfuse MOVs are designed to suppress transient voltages such as lightning and other high level transients found in industrial and AC line applications. For the purposes of shunting energy for an AED applied to a subject implanted with an EID the peak energies vary but range from about 100 J to about 200 J.

For example, given a nominal 1500V defibrillation energy delivered via an AED a varistor such as an MOV coupled to a conductive feedthrough pin that passes through the housing or shield of an EID will allow up to 1500V to defibrillate the heart and any energy over 1500V will be partially shunted. Thus, the electrical voltage appearing across the terminals of an EID will be limited to less than about 1600V thereby protecting the EID circuitry while allowing external defibrillation therapy to proceed essentially unimpeded.

The present invention generally relates to implantable medical devices, particularly implantable (cardioverter) defibrillators that are entirely implanted subcutaneously and, more particularly, have no leads or electrodes contacting the heart or extending into the thoracic cavity.

Apparatuses and methods are disclosed relating to various types of EID's with geometries, shapes and sizes adapted for subcutaneous or submuscularimplant. In a prophylactic application, for example, some embodiments form EID systems that can be placed completely in the subcutaneous or submuscular position without the need to place leads or electrodes in the vasculature of the patient. One set of embodiments of the invention provides a variety of configurations for delivering cardioversion/defibrillation therapy with a vector of energy controlled by operative circuitry of a non-active-can type EID. In one form of the invention, the EID housing can be conveniently implanted in a surgically-created subcutaneous or submuscular pocket formed over or near a portion of the cardiac notch, or sternum of a patient and adjacent a portion of pectoralis major.

In yet another embodiment, the EID may be implanted in a pocket formed adjacent a portion of the external abdominal oblique. In another embodiment, the EID housing may be implanted in a pocket formed adjacent a portion of the serratus anterior.

In one embodiment, the EID electrically couples to one or more elongated, coil-type high voltage electrodes with the electrodes disposed in a location providing defibrillation vectors covering adequate mass of myocardial tissue to achieve defibrillation and deliver pacing therapy. Specifically, leads may be substantially implanted adjacent a portion of the external abdominal oblique; adjacent the cardiac notch; adjacent a portion of the serratus anterior; and adjacent a portion of the latissimus dorsi.

In one embodiment, more than one high voltage electrodes are implemented with the EID connected to all electrodes. The one or more high voltage electrodes may include a set of coil electrodes disposed in an orientation relative to a patient's heart that provides several different therapy delivery vectors therebetween.

### BRIEF DESCRIPTION OF THE DRAWINGS

These and other features of the present invention will be appreciated as the same becomes better understood by reference to the following detailed description of the preferred embodiment of the invention when considered in connection with the accompanying drawings, in which like numbered reference numbers designate like parts throughout the figures thereof.

FIG. 1A depicts a multi-planar view of an EID of a first embodiment of the present invention.

FIG. 1B illustrates an EID of the first embodiment implanted in a patient.

FIG. 2A illustrates a multi-planar view of an EID of a second embodiment of the present invention.

FIG. 2B illustrates an EID of the second embodiment implanted in a patient.

FIG. 3A illustrates a multi-planar view of a third embodiment of an EID in accordance with the present invention.

FIG. 3B illustrates the EID of the third embodiment implanted in a patient.

FIG. 4A illustrates the EID of the fourth embodiment in accordance with the present invention.

FIG. 4B illustrates the EID of the fourth embodiment implanted in a patient.

FIG. 4C illustrates a cross-sectional view of a cable connecting the two parts of an EID of the fourth embodiment in accordance with the present invention.

FIGS. 4D, 4E and 4F illustrate a cross-sectional view of a patient taken through the thoracic cavity and center of the heart showing the deployment and arrangement of the fourth embodiment EID in accordance with the present invention.

FIG. 5A illustrates a multi-planar view of an EID in accordance with a fifth embodiment of the present invention.

FIGS. 5B and 5C illustrate a cross-sectional view of a patient taken through the thoracic cavity and center of the heart with the deployment and arrangement of the EID, and the EID of the fifth embodiment implanted in a patient, respectively.

FIG. 6A illustrates a multi-planar view of another EID embodiment.

FIGS. 6B and 6C illustrate perspective views of an EID showing major internal piece parts of a generic embodiment.

FIG. 7 illustrates a block diagram of the circuitry of an exemplary EID.

FIG. 8 illustrates a schematic indicating the relative electrical connections of a EID according to the invention as well as the representative couplings of a pair of surface-paddle electrodes of an external defibrillator.

### DETAILED DESCRIPTION OF THE INVENTION

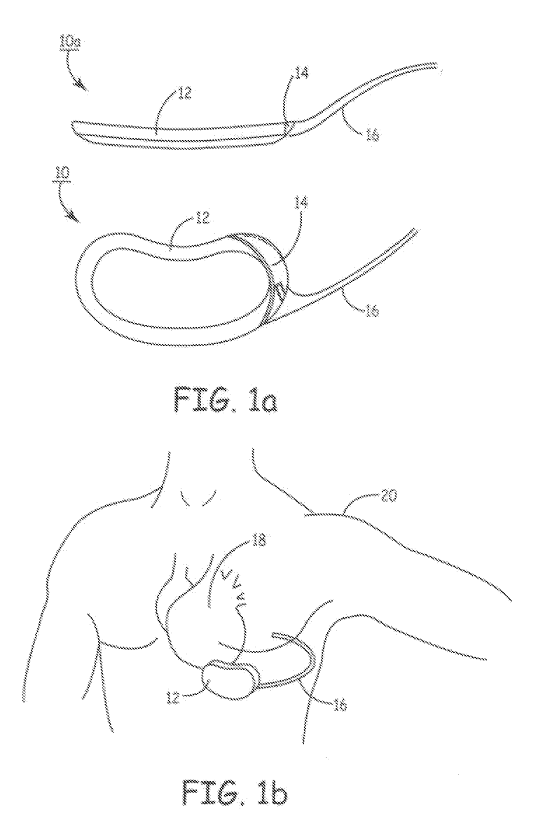

FIG. 1A depicts a multi-planar view of a first embodiment of the present invention. EID 12 is an ovoid, substantially, kidney-shaped housing with connector 14 for attaching a subcutaneous sensing and cardioversion/defibrillation therapy delivery lead 16. EID 12 may be constructed of stainless steel, titanium or ceramic as described in U.S. Pat. No. 4,180,078 “Lead Connector for a Body Implantable Stimulator” to Anderson and U.S. Pat. No. 5,470,345 “Implantable Medical Device with Multi-layered Ceramic Enclosure” to Hassler, et al. The electronics circuitry of EID 10 (described herein pertaining to FIG. 21) may be incorporated on a polyamide flex circuit, printed circuit board (PCB) or ceramic substrate with integrated circuits packaged in leadless chip carriers and/or chip scale packaging (CSP). In one of the views, the concave construction of EID 12 is illustrated. The minor concavity of the housing of EID 12 follows the natural curve of the patient's median ribcage at about the cardiac notch. The central curved depression shown in frontal elevation view 10 is ergonomically aligned to minimize patient discomfort when seated, bending over and/or during normal torso movement.

EID 12 is shown coupled to subcutaneous lead 16. At connector block 14, the crescent-shaped connector block 14 enables a reliable curvilinear connection between lead 16 and the curved edge of EID 12. Lead 16, like the other leads discussed below, includes an elongated lead body carrying conventional, mutually insulated conductors, each coupled to an electrode.

FIG. 1B illustrates EID 12 implanted in patient 20. Specifically, lead 16 is advanced adjacent the cardiac notch and tunneled subcutaneously from the median implant pocket of EID 12 laterally and posterially to the patient's back to a location opposite the heart such that the heart 18 is disposed between the EID 12 and the distal end of subcutaneous lead 16. The implant location of EID 12 and lead 16 is typically subcutaneously above the external abdominal oblique. The distal end of lead 16 is tunneled above the external oblique muscle extending over to a portion of the latissimus dorsi.

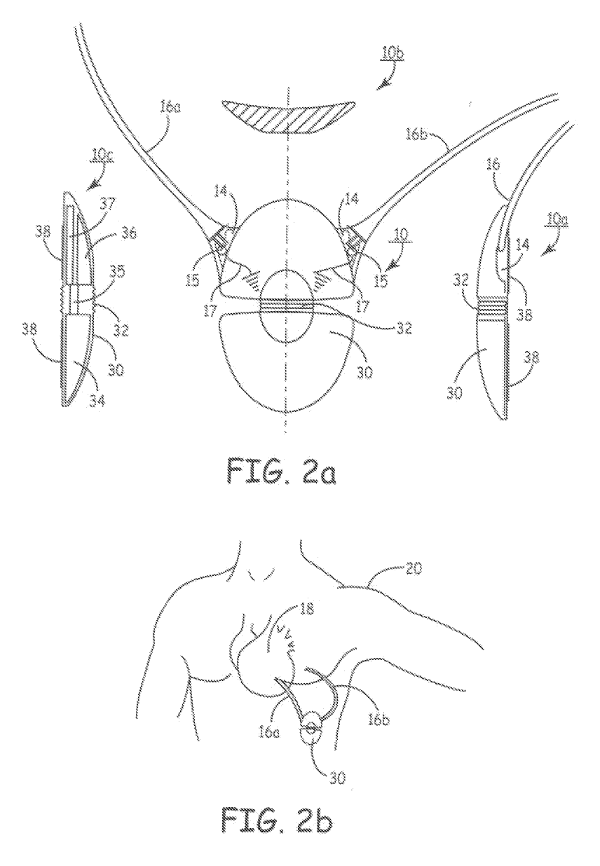

FIG. 2A is a multi-planar view of EID 30, a second embodiment of the present invention. EID 30 is a convex, flexible ovaloid-shaped housing with connectors 14 (two shown) for attaching a pair of subcutaneous sensing and cardioversion/defibrillation therapy delivery leads 16A and 16b. According to the invention one or more components capable of limiting electrical voltage, such as a metal oxide varistor 15 couples to the leads 16a and 16b and via a conductor 17 to a source of common electrical reference voltage (e.g., the housing of EID 30).

EID 30 may be constructed of stainless steel, titanium or ceramic. View 10A is a side view of EID 30 showing the tapered housing 30, a mid-line flexible joint 32, connector 14, lead 16 and active can electrode 38. The active can electrode 38 allows sensing and cardioversion, defibrillation and/or pacing therapy delivery between the EID 30 and one or both leads 16A or 16b. The jointed housing 30 allows physician flexibility in selecting implant locations and accommodates variances in size and weight of patients for implant. Additionally, the flexible housing provides less patient discomfort in sitting, bending over and/or during normal torso movement because the configurations allows dynamic adjustment to the patient's dynamic and muscular movements. View 10b is a top cut-away view of the EID 30 showing the convex construction that promotes ease of subcutaneous implant by following the natural curve of the patient's lateral ribcage. View 10c is a vertical cross section of EID 30 showing internal components that will be described in more detail hereinbelow. Shown in this view are battery 36, electronics module 37, high voltage capacitors 34, flex circuit 35 and flexible housing joint 32.

FIG. 2B illustrates implant position of EID 30 and leads 16A and 16B according to a second embodiment of the invention. EID 30 is implanted subcutaneously over a portion of the external oblique muscle laterally outside the 20^{th} ribcage of patient 20. Lead 16A is tunneled subcutaneously from the lateral implant pocket of EID 30 anterially and medially to the cardiac notch. Further, lead 16b is tunneled posterially adjacent the latissimus dorsi, to the patient's back to a location opposite the heart such that the heart 18 is disposed between the distal end of subcutaneous lead 16A and the distal end of subcutaneous lead 16b.

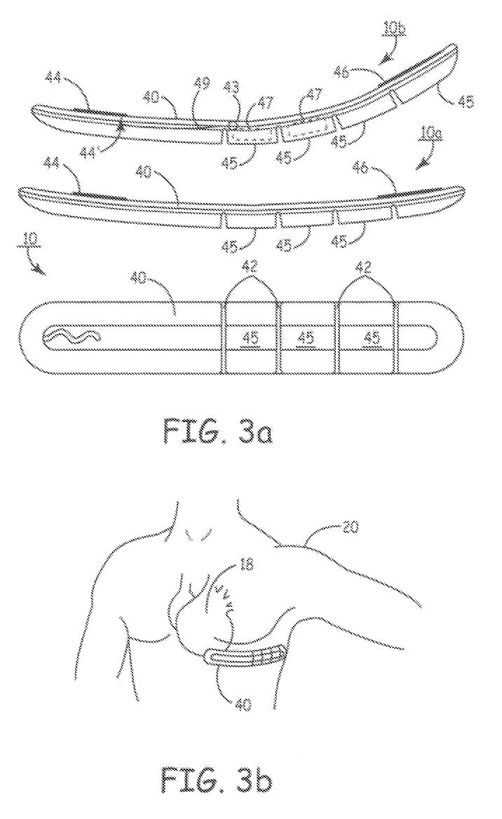

FIG. 3A is a multi-planar view of a third embodiment of EID 40. EID 40 is an elongated slender ellipsoid with sections of partially articulating dynamic segments having surface mounted subcutaneous sensing and cardioversion/defibrillation therapy delivery electrodes 44 and 46. EID 40 may be constructed of stainless steel, titanium or ceramic or equivalent. View 10A is a top view of EID 40 showing the segmented construction (at 42). One or more of the segmented portions 45 can be adapted to house, for instance, high voltage defibrillation circuitry 47. According to the invention, an energy limiting component (or components) 43 can couple to an electrical reference (e.g., a ground or common electrical potential for the device, such as a portion of the metallic housing) and to a conductor 49 that couples to a high voltage electrode 44 (at 44′). Electrodes 44 and 46 located at opposite ends of EID 40 are typically 100 mm² to 1000 mm². View 10b is a further top view showing the dynamic flexibility of EID 40 in which it assumes a dynamically adjustable, compressive and tensile opposing surfaces when implanted outside the thoracic cavity over the ribs. Specifically, in its normal position, EID 40 is substantially flat both at the top and bottom surfaces. However, when implanted, EID 40 dynamically forms a concave and convex surface at the flat top and segmented bottom surfaces when tunneled into the subcutaneous regions of the thoracic cavity above the ribs or the intercostals region therebetween. As illustrated in FIG. 3B, EID 40 dynamically adjusts to wrap around the ribcage with electrode 44 anterior to the cardiac notch and the EID 40 is positioned such that electrode 46 is laterally located in opposition to electrode 46 thereby positioning heart 18 between the electrodes. The dynamic configurability of EID 40 creates an external surface that is convex and slightly bent in two directions and at the same time twisted on its long axis to closely fit over the ribs.

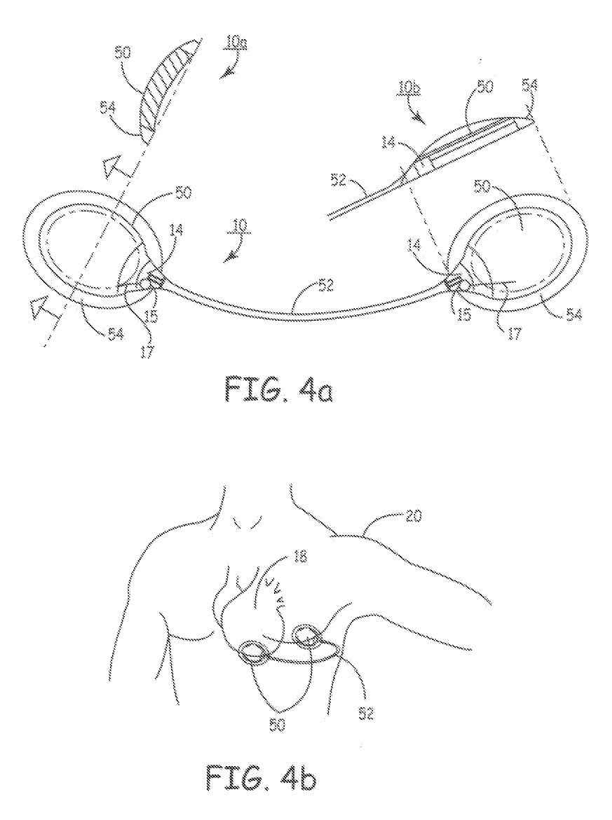

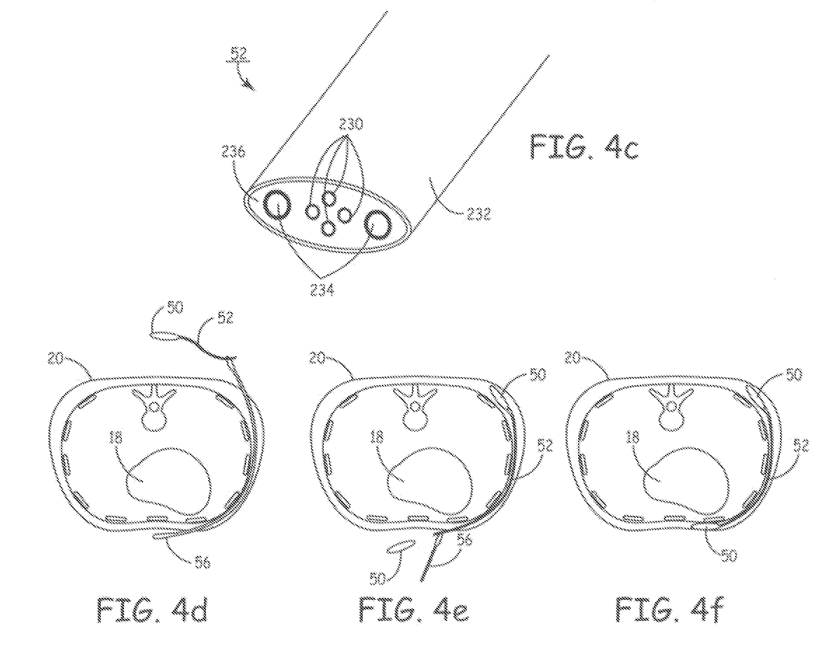

FIG. 4A is an illustration of the fourth embodiment of the present invention. EID 50 housings are connected by an interconnecting lead 52 containing power, control, sensing and therapy delivery conductors. The EID 50 contains integrated subcutaneous sensing and cardioversion/defibrillation therapy delivery electrodes. EID 50 may be constructed of stainless steel, titanium or ceramic. View 10A is a cross sectional view through one of the EID 50 housings showing the concave inner surface to enable un-obtrusive subcutaneous implant because the oval profile of EID 50 is designed to follow the natural curve of the patient's median cardiac notch and posterior ribcage. Integrated electrodes (not shown) located on the inner surfaces of EID 50 are typically 100 mm² to 1000 mm² in active area. View 10b is a top view of EID 50 showing a convex domed top and a substantially flat bottom. According to the invention one or more components capable of limiting electrical current, such as a metal oxide varistor 15 couples to the leads 16a and 16b and via a conductor 17 to a source of common electrical reference voltage (e.g., the housing of EID 30).

FIG. 4B illustrates EID 50 implanted in patient 20. Specifically, EID 50 is implanted outside the ribcage with a first EID 50 housing anterior to the cardiac notch and the other EID 50 housing tunneled and positioned posterially in relation to heart 18.

FIG. 4C illustrates a cross-sectional view of the interconnecting cable 52. The outer sheath of cable 52 consists of a urethane or equivalent sheath 232 with an inner insulation 236 of HP Silicone. The power, control and sensing conductors 230 are wrapped with ETFE while the defibrillation conductors 234 are constructed of Ag/MP35N and wrapped with ETFE and reinforced with tensile material.

FIGS. 4D, 4E and 4F illustrate cross-sectional views taken through the thoracic cavity and center of the heart showing the deployment and implant of EID 50. FIG. 4D shows a tunneling tool 56 entering the patient's body 20 at a first incision anterior to the cardiac notch, tunneled laterally and posterially to exit at a second incision in the patient's back adjacent a portion of the latissimus dorsi. The EID 50 and interconnecting cable 52 are attached to the tunneling tool 56, which is retracted, and the EID 50 and cable 52 are pulled into a posterior implant location as shown in FIG. 4E. The second housing of EID 50 is attached to the interconnecting cable 52 and placed into an implant pocket anterior to the cardiac notch as shown in FIG. 4F.

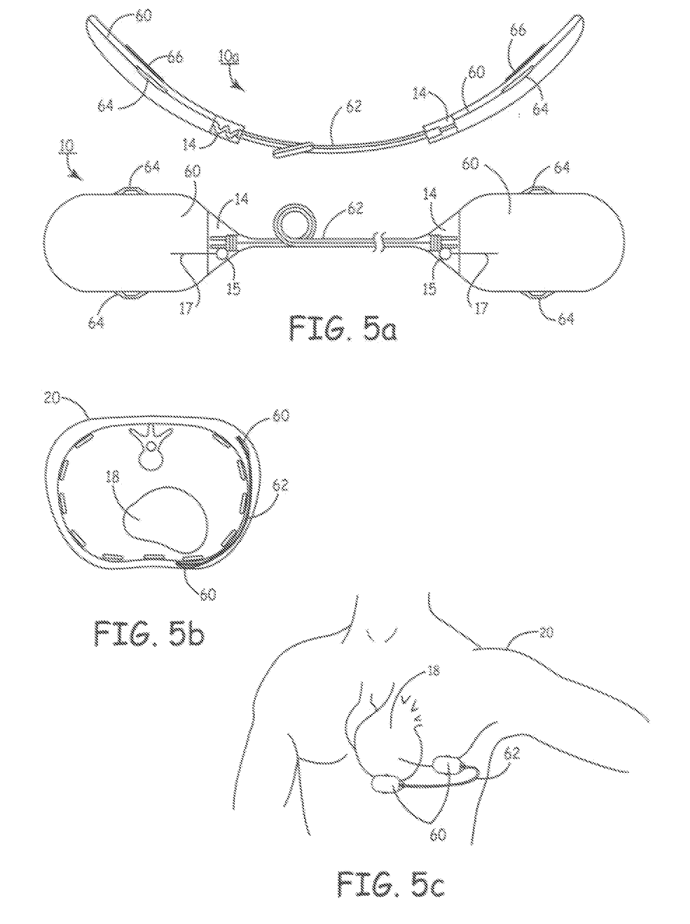

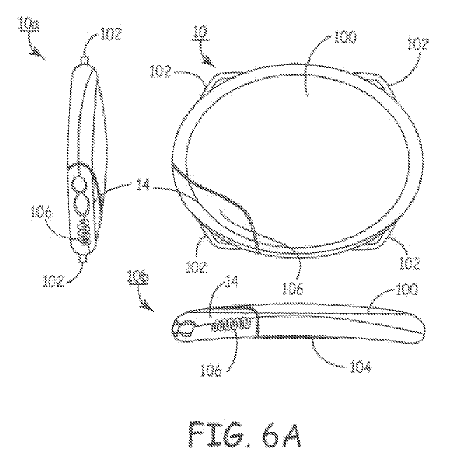

FIG. 5A illustrates a fifth embodiment of the present invention. EID 50 consists of two rounded beetle-shaped housings connected by an interconnecting lead 62. The EID 60 may be constructed of stainless steel, titanium or ceramic. Excess length and a strain relief loop are provided in cable 62. The EID 60 contains integrated subcutaneous sensing and cardioversion/defibrillation therapy delivery electrodes 66. Suture loops 64 are provided on each housing to enable the fixation of each housing in a predetermined location for proper stimulation and to prevent device migration. As is shown in the top view, EID 60 housing includes a concave inner surface to enable a compliant subcutaneous movement by the canisters following the natural curve of the patient's median cardiac notch and posterior ribcage. Integrated electrodes 66 located on the inner surfaces of canisters 60 are typically 100 mm² to 1000 mm² in active area. According to the invention one or more components capable of limiting electrical current, such as a metal oxide varistor 15 couples to the leads 16a and 16b and via a conductor 17 to a source of common electrical reference voltage (e.g., the housing of EID 30). The component 15 can be disposed upon a portion of a hybrid circuit board (not shown) and in order to increase temperature dissipation, a layer or block of a material capable of functioning as a heat sink can be applied under the component 15. In one embodiment, a layer of copper is disposed under the component 15 and electrically isolated from the other circuitry and active components of the EID 30. In addition, known types of capacitive filtering components can be used in addition to the component 15. In one form of the invention, discoidal capacitors are integrated into a feedthrough assembly to reduce or eliminate electronic interference from entering the housing 60.

FIG. 5B illustrates a cross-sectional view through the thoracic cavity and the center of the heart 18 showing the implant location for EID 60. Specifically, a first housing of EID 60 is implanted anterior to the cardiac notch and a second housing of EID 60 located posterially. Interconnecting cable 62 containing power, control, sensing and therapy delivery conductors is located between the EID 60 housing as shown.

FIG. 5C illustrates EID 60 implanted in patient 20. As discussed hereinabove, EID 60 is subcutaneously implanted with the two housings carrying exposed large surface electrodes. The positioning is such that a major potion of the myocardium of heart 18 is located between the two electrodes 66 on each housing of EID 60.

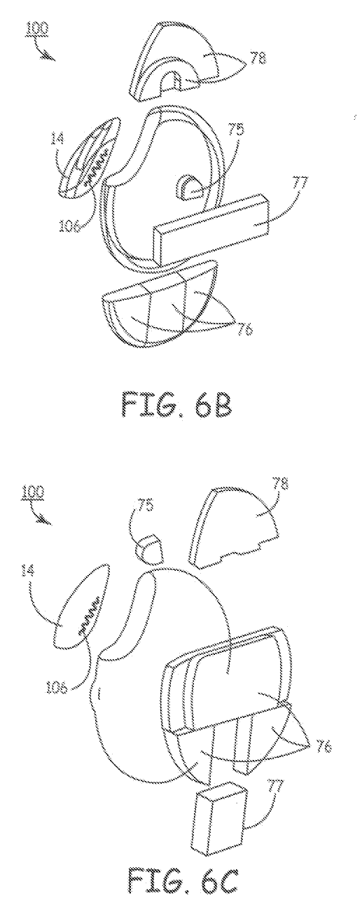

FIG. 6A is a plan side view of a subcutaneous cardioverter-defibrillator 10 of a ninth embodiment of the present invention. Canister 100 is an ovaloid-shaped housing with a connector 14 for attaching 1 or 2 subcutaneous sensing and cardioversion/defibrillation therapy delivery leads. This design allows great flexibility in device placement and location. Canister 100 may be constructed of stainless steel, titanium or ceramic. The electronics circuitry of subcutaneous cardioverter-defibrillator 10 (described later in relation to FIG. 21) may be incorporated on a polyamide flex circuit, printed circuit board (PCB) or ceramic substrate with integrated circuits packaged in leadless chip carriers and/or chip scale packaging (CSP). View 10A is an end view of subcutaneous cardioverter-defibrillator 100 showing the connector 14, suture loops 102 (2 shown) and antenna 106. Suture loops 102 are provided on housing 100 to allow the fixation of housing in a fixed pocket location for proper stimulation and to prevent device migration.

FIG. 6B is a plan view showing the component parts/elements of the EID 100 of FIG. 6A. Components shown include, battery 77, electronics module 76, tantalum capacitors 76 (3 shown), transformer 75, antenna 106 and connector 14.

FIG. 6C is a perspective view showing an alternative embodiment of the major piece parts/elements of the EID 100 of FIG. 6A. Components shown include, battery 77, electronics module 78, aluminum capacitors 76 (4 shown), transformer 75, antenna 106 and connector 14.

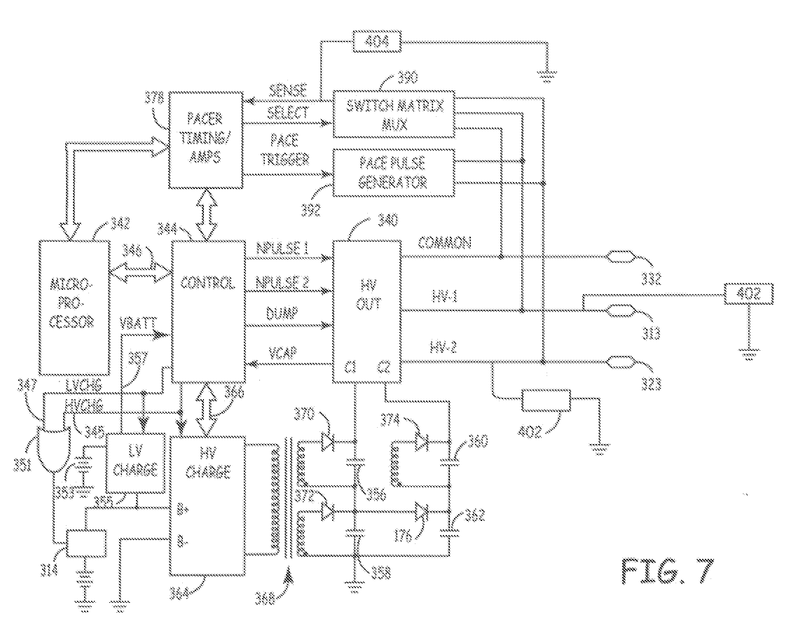

The electronic circuitry employed in the EID (as described above in relation to the various embodiments shown in FIG. 1-15) can take any of the known forms that detect a tachyarrhythmia from the sensed EGM and provide cardioversion/defibrillation shocks as well as post-shock pacing as needed. A simplified block diagram of such circuitry adapted to function employing the first and second and, optionally, the third cardioversion-defibrillation electrodes as well as the EGM sensing and pacing electrodes described above is set forth in FIG. 7. It will be understood that the simplified block diagram does not show all of the conventional components and circuitry of such ICDs including digital clocks and clock lines, low voltage power supply and supply lines for powering the circuits and providing pacing pulses or telemetry circuits for telemetry transmissions between the ICD and an external programmer or monitor.

FIG. 7 illustrates the electronic circuitry, low voltage and high voltage batteries within the hermetically sealed housings. The low voltage battery 353 is coupled to a power supply (not shown) that supplies power to the ICD circuitry and the pacing output capacitors to supply pacing energy in a manner well known in the art. The low voltage battery can comprise one or two conventional LiCFₓ, LiMnO₂ or LiI₂ cells. The high voltage battery 312 can comprise one or two conventional LiSVO or LiMnO₂ cell.

In FIG. 7, EID functions are controlled by means of stored software, firmware and hardware that cooperatively monitor the EGM, determine when a cardioversion-defibrillation shock or pacing is necessary, and deliver prescribed cardioversion-defibrillation and pacing therapies. The block diagram of FIG. 7 incorporates circuitry set forth in commonly assigned U.S. Pat. No. 5,163,427 “Apparatus for Delivering Single and Multiple Cardioversion and Defibrillation Pulses” to Keimel; U.S. Pat. No. 5,188,105 “Apparatus and Method for Treating a Tachyarrhythmia” to Keimel and U.S. Pat. No. 5,314,451 “Replaceable Battery for Implantable Medical Device” to Mulier for selectively delivering single phase, simultaneous biphasic and sequential biphasic cardioversion-defibrillation shocks typically employing an ICD IPG housing electrode coupled to the COMMON output 332 of high voltage output circuit 340 and one or two cardioversion-defibrillation electrodes disposed in a heart chamber or cardiac vessel coupled to the HVI and HV-2 outputs (313 and 323, respectively) of the high voltage output circuit 340. The circuitry of the subcutaneous EID of the present invention can be made simpler by adoption of one such cardioversion-defibrillation shock waveform for delivery simply between the first and second cardioversion-defibrillation electrodes 313 and 323 coupled to the HV-I and HV-2 outputs respectively. Or, the third cardioversion-defibrillation electrode 332 can be coupled to the COMMON output as depicted in FIG. 7 and the first and second cardioversion-defibrillation electrodes 313 and 323 can be electrically connected in to the HV-1 and the HV-2 outputs, respectively, as depicted in FIG. 7.

The cardioversion-defibrillation shock energy and capacitor charge voltages can be intermediate to those supplied by ICDs having at least one cardioversion-defibrillation electrode in contact with the heart and most AEDs having cardioversion-defibrillation electrodes in contact with the skin. The typical maximum voltage necessary for ICDs using most biphasic waveforms is approximately 750 Volts with an associated maximum energy of approximately 40 Joules. The typical maximum voltage necessary for AEDs is approximately 2000-5000 Volts with an associated maximum energy of approximately 200-360 Joules depending upon the model and waveform used. The ICD of the present invention uses maximum voltages in the range of about 700 to about 3150 Volts and is associated with energies of about 25 Joules to about 210 Joules. The total high voltage capacitance could range from about 50 to about 300 microfarads.

Such cardioversion-defibrillation shocks are only delivered when a malignant tachyarrhythmia, e.g., ventricular fibrillation is detected through processing of the far field cardiac EGM employing one of the available detection algorithms known in the ICD art.

In FIG. 7, pacer timing/sense amplifier circuit 378 processes the far field EGM SENSE signal that is developed across a particular EGM sense vector defined by a selected pair of the electrodes 332, 313 and, optionally, electrode 323 if present as noted above. The selection of the sensing electrode pair is made through the switch matrix/MUX 390 in a manner disclosed in the commonly assigned U.S. Pat. No. 5,331,966 “Subcutaneous Multi-Electrode Sensing System, Method and Pacer” to Bennett, et al patent to provide the most reliable sensing of the EGM signal of interest, which would be the R wave for patients who are believed to be at risk of ventricular fibrillation leading to sudden death. The far field EGM signals are passed through the switch matrix/MUX 390 to the input of a sense amplifier in the pacer timing/sense amplifier circuit 378. Bradycardia is typically determined by an escape interval timer within the pacer timing circuit 378 or the timing and control circuit 344, and pacing pulses that develop a PACE TRIGGER signal applied to the pacing pulse generator 392 when the interval between successive R-waves exceeds the escape interval. Bradycardia pacing is often temporarily provided to maintain cardiac output after delivery of a cardioversion-defibrillation shock that may cause the heart to slowly beat as it recovers function.

Detection of a malignant tachyarrhythmia is determined in the timing and control circuit 344 as a function of the intervals between R-wave sense event signals that are output from the pacer timing/sense amplifier circuit 378 to the timing and control circuit 344.

Certain steps in the performance of the detection algorithm criteria are cooperatively performed in a microcomputer 342, including microprocessor, RAM and ROM, associated circuitry, and stored detection criteria that may be programmed into RAM via a telemetry interface (not shown) conventional in the art. Data and commands are exchanged between microcomputer 342 and timing and control circuit 344, pacer timing/amplifier circuit 378, and high voltage output circuit 340 via a bi-directional data/control bus 346. The pacer timing/amplifier circuit 378 and the timing and control circuit 344 are clocked at a slow clock rate. The microcomputer 342 is normally asleep, but is awakened and operated by a fast clock by interrupts developed by each it-wave sense event or on receipt of a downlink telemetry programming instruction or upon delivery of cardiac pacing pulses to perform any necessary mathematical calculations, to perform tachycardia and fibrillation detection procedures, and to update the time intervals monitored and controlled by the timers in pace/sense circuitry 378. The algorithms and functions of the microcomputer 342 and timer and control circuit 344 employed and performed in detection of tachyarrhythmias are set forth, for example, in commonly assigned U.S. Pat. No. 5,991,656 “Prioritized Rule Based Apparatus for Diagnosis and Treatment of Arrhythmias” to Olson, et al and U.S. Pat. No. 5,193,535 “Method and Apparatus for Discrimination of Ventricular Tachycardia from Ventricular Fibrillation and for Treatment Thereof” to Bardy, et al, for example. Particular algorithms for detection of ventricular fibrillation and malignant ventricular tachycardias can be selected from among the comprehensive algorithms for distinguishing atrial and ventricular tachyarrhythmias from one another and from high rate sinus rhythms that are set forth in the '656 and '535 patents.

The detection algorithms are highly sensitive and specific for the presence or absence of life threatening ventricular arrhythmias, e.g., ventricular tachycardia (V-TACH) and ventricular fibrillation (V-FIB). Another optional aspect of the present invention is that the operational circuitry can detect the presence of atrial fibrillation (A FIB) as described in Olson, W. et al. “Onset And Stability For Ventricular Tachyarrhythmia Detection in an Implantable Cardioverter and Defibrillator,” Computers in Cardiology (1986) pp. 167-170. Detection can be provided via R-R Cycle length instability detection algorithms. Once A-FIB has been detected, the operational circuitry will then provide QRS synchronized atrial cardioversion/defibrillation using the same shock energy and wave shapes used for ventricular cardioversion/defibrillation.

Operating modes and parameters of the detection algorithm are programmable and the algorithm is focused on the detection of V-FIB and high rate V-TACH (>240 bpm).

Although the EID of the present invention may rarely be used for an actual sudden death event, the simplicity of design and implementation allows it to be employed in large populations of patients at modest risk with modest cost by medical personnel other than electrophysiologists. Consequently, the EID of the present invention includes the automatic detection and therapy of the most malignant rhythm disorders. As part of the detection algorithm's applicability to children, the upper rate range is programmable upward for use in children, known to have rapid supraventricular tachycardias and more rapid V-FIB.

When a malignant tachycardia is detected, high voltage capacitors 356, 358, 360, and 362 are charged to a pre-programmed voltage level by a high-voltage charging circuit 364. It is generally considered inefficient to maintain a constant charge on the high voltage output capacitors 356, 358, 360, 362. Instead, charging is initiated when control circuit 344 issues a high voltage charge command HVCHG delivered on line 345 to high voltage charge circuit 364 and charging is controlled by means of bi-directional control/data bus 366 and a feedback signal VCAP from the HV output circuit 340. High voltage output capacitors 356, 358, 360 and 362 may be of film, aluminum electrolytic or wet tantalum construction.

The negative terminal of high voltage battery 312 is directly coupled to system ground. Switch circuit 314 is normally open so that the positive terminal of high voltage battery 312 is disconnected from the positive power input of the high voltage charge circuit 364. The high voltage charge command HVCHG is also conducted via conductor 349 to the control input of switch circuit 314, and switch circuit 314 closes in response to connect positive high voltage battery voltage EXT B+ to the positive power input of high voltage charge circuit 364. Switch circuit 314 may be, for example, a field effect transistor (FET) with its source-to-drain path interrupting the EXT B+ conductor 318 and its gate receiving the HVCHG signal on conductor 345. High voltage charge circuit 364 is thereby rendered ready to begin charging the high voltage output capacitors 356, 358, 360, and 362 with charging current from high voltage battery 312.

High voltage output capacitors 356, 358, 360, and 362 may be charged to very high voltages, e.g., 700-3150V, to be discharged through the body and heart between the selected electrode pairs among first, second, and, optionally, third subcutaneous cardioversion-defibrillation electrodes 313, 332, and 323. In accordance with certain aspects of the present invention a metal oxide varistor 400,402 electrically couples intermediate a high voltage electrode (e.g., 313,323) and a source of reference voltage (e.g., internal circuitry of the EID). Thus, in the event that external defibrillation therapy is delivered to a patient having an EID, the defibrillation energy passes to the myocardium and does not shunt to the EID thereby possibly damaging the EID and/or limiting the defibrillation energy delivered to the patient. Another voltage-limiting component 404 can be placed across the pacing sensing amplifier(s) 378 used to sense far field cardiac wavefronts. This component 404 thus protects the amplifiers 378 from damage during application of external defibrillation therapy.

The details of the voltage charging circuitry are also not deemed to be critical with regard to practicing the present invention; one high voltage charging circuit believed to be suitable for the purposes of the present invention is disclosed. High voltage capacitors 356, 358, 360, and 362 are charged by high voltage charge circuit 364 and a high frequency, high-voltage transformer 368 as described in detail in commonly assigned U.S. Pat. No. 4,548,209 “Energy Converter for Implantable Cardioverter” to Wielders, et al. Proper charging polarities are maintained by diodes 370, 372, 374 and 376 interconnecting the output windings of high-voltage transformer 368 and the capacitors 356, 358, 360, and 362. As noted above, the state of capacitor charge is monitored by circuitry within the high voltage output circuit 340 that provides a VCAP, feedback signal indicative of the voltage to the timing and control circuit 344. Timing and control circuit 344 terminates the high voltage charge command HVCHG when the VCAP signal matches the programmed capacitor output voltage, i.e., the cardioversion-defibrillation peak shock voltage.

Timing and control circuit 344 then develops first and second control signals NPULSE 1 and NPULSE 2, respectively, that are applied to the high voltage output circuit 340 for triggering the delivery of cardioverting or defibrillating shocks. In particular, the NPULSE 1 signal triggers discharge of the first capacitor bank, comprising capacitors 356 and 358. The NPULSE 2 signal triggers discharge of the first capacitor bank and a second capacitor bank, comprising capacitors 360 and 362. It is possible to select between a plurality of output pulse regimes simply by modifying the number and time order of assertion of the NPULSE 1 and NPULSE 2 signals. The NPULSE 1 signals and NPULSE 2 signals may be provided sequentially, simultaneously or individually. In this way, control circuitry 344 serves to control operation of the high voltage output stage 340, which delivers high energy cardioversion-defibrillation shocks between a selected pair or pairs of the first, second, and, optionally, the third cardioversion-defibrillation electrodes 313, 323, and 332 coupled to the HV-1, HV-2 and optionally to the COMMON output as shown in FIG. 7.

Thus, EID 10 monitors the patient's cardiac status and initiates the delivery of a cardioversion-defibrillation shock through a selected pair or pairs of the first, second and third cardioversion-defibrillation electrodes 313, 323 and 332 in response to detection of a tachyarrhythmia requiring cardioversion-defibrillation. The high HVCHG signal causes the high voltage battery 312 to be connected through the switch circuit 314 with the high voltage charge circuit 364 and the charging of output capacitors 356, 358, 360, and 362 to commence. Charging continues until the programmed charge voltage is reflected by the VCAP signal, at which point control and timing circuit 344 sets the HVCHG signal low terminating charging and opening switch circuit 314. Typically, the charging cycle takes only fifteen to twenty seconds, and occurs very infrequently. The EID 10 can be programmed to attempt to deliver cardioversion shocks to; the heart in the manners described above in timed synchrony with a detected R-wave or can be programmed or fabricated to deliver defibrillation shocks to the heart in the manners described above without attempting to synchronize the delivery to a detected R-wave. Episode data related to the detection of the tachyarrhythmia and delivery of the cardioversion-defibrillation shock can be stored in RAM for uplink telemetry transmission to an external programmer as is well known in the art to facilitate in diagnosis of the patient's cardiac state. A patient receiving the EID 10 on a prophylactic basis would be instructed to report each such episode to the attending physician for further evaluation of the patient's condition and assessment for the need for implantation of a more sophisticated and long-lived EID.

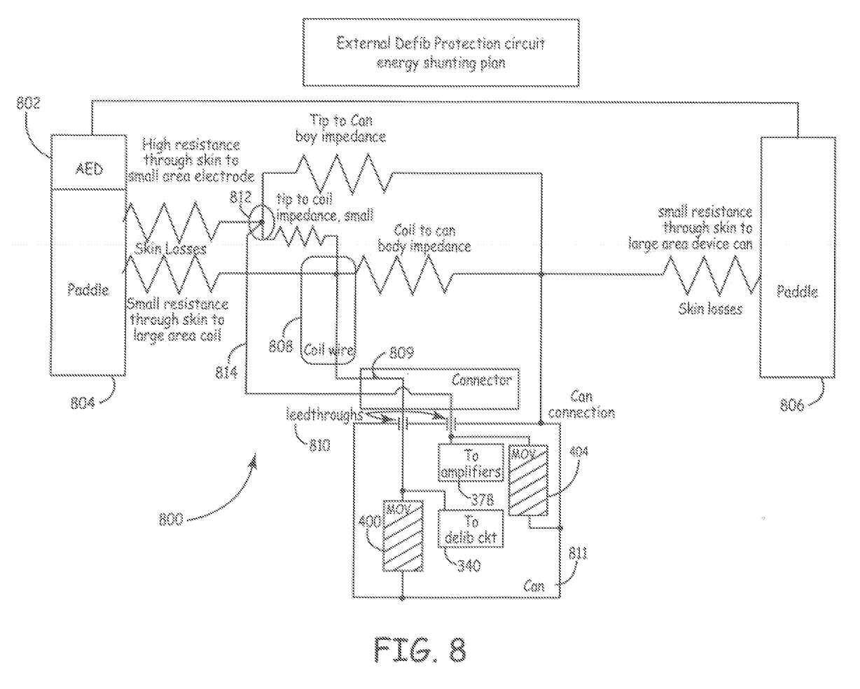

FIG. 8 illustrates a schematic indicating the relative electrical connections of an EID 800 according to the invention as well as the representative couplings of a pair of surface-paddle electrodes of an external defibrillator 802 (e.g., an automated external defibrillator or a manually operated emergency technician-operated defibrillator) and the paddle electrodes 804,806 coupled thereto. In FIG. 8 various inherent sources of electrical impedances are represented schematically (e.g., interface between a patient's skin and the paddle electrodes 804,806 as well as the inter-electrode impedances). In the embodiment depicted in FIG. 8, a single high-voltage coil electrode 808 couples via elongated conductor 809 to high voltage defibrillation circuitry 340 disposed within the EID 800. The conductor 809 enters the shield or “can” 811 of the EID via a hermetically sealed conductive feedthrough 810. Also coupled to this interconnected circuit is a voltage-limiting component 400 (e.g., a metal oxide varistor) which in turn couples to a source of electrical reference such as the metallic shield or “can” 811.

An electrode used to sense far field cardiac activity, such as the coil electrode 808 and/or an additional electrode 812 couples via conductor 814 to amplifier circuitry 378 and conductor 814 also couples to a voltage-limiting component 404 (e.g., a metal oxide varistor) according to the invention. As is known in the art the feedthrough(s) 810 typically provide electrical insulation from the traditionally conductor can 811 and are oftentimes disposed intermediate the can and a connector component that provides a reliable means of coupling the conductors 809,814 to said feethroughs 810.

Also, in one embodiment a surface portion of a metallic housing that faces away from a heart for an EID according to the invention can be coated with dielectric material (or otherwise insulated) and a portion facing the heart can be actively electrified. Thus, the non-conductive surface acts to reduce the energy dissipated by the MOV which allows a relatively smaller MOV to be used.

It will be apparent from the foregoing that while particular embodiments of the invention have been illustrated and described, various modifications can be made without departing from the spirit and scope of the invention. Accordingly, it is not intended that the invention be limited, except as by the appended claims.

1. A subcutaneous extra-cardiac implantable medical device (EID), comprising:
a housing comprised at least in part of an electrically conductive material;
at least one active circuit disposed in the housing;
a source of electrical energy coupled to the at least one active circuit;
an insulated elongated conductive member coupled to the at least one active circuit; and
a voltage-limiting component coupled to the elongated conductive member intermediate the at least one active circuit and the source of electrical energy.

2. A subcutaneous EID according to claim 1, wherein the housing comprises a biocompatible metallic material.

3. A subcutaneous EID according to claim 2, wherein the housing comprises one of titanium and stainless steel.

4. A subcutaneous EID according to claim 1, wherein the source of electrical energy comprises at least one capacitor.

5. A subcutaneous EID according to claim 4, wherein the capacitor comprises a valve metal-based capacitor.

6. A subcutaneous EID according to claim 5, wherein the valve metal-based capacitor comprises one of a tantalum-based capacitor and an aluminum-based capacitor.

7. A subcutaneous EID according to claim 1, wherein the voltage-limiting component comprises a varistor.

8. A subcutaneous EID according to claim 7, wherein the varistor comprises a metal oxide varistor (MOV).

9. A subcutaneous EID according to claim 8, wherein the MOV is configured to shunt external electrical energy having a magnitude of over about 1000 volts.

10. A subcutaneous EID according to claim 9, wherein the MOV is configured to shunt electrical energy having a magnitude of over about 1500 volts.

11. A subcutaneous EID according to claim 1, wherein the EID comprises an extra-cardiac implantable defibrillator.

12. A subcutaneous EID according to claim 11, wherein the EID is adapted for implantation in one of a submuscular location and a location adjacent an intercostal location.

13. A subcutaneous EID according to claim 1, wherein the insulated elongated conductive member comprises a medical electrical lead having a high voltage defibrillation electrode operatively coupled thereto.

14. A subcutaneous EID according to claim 13, wherein the high voltage defibrillation electrode comprises one of a coil-type electrode and a patch-type electrode.

15. A method of shunting electrical energy from an extra-cardiac implantable defibrillator (EID) when the is subjected to a high voltage defibrillation therapy, comprising:
operatively coupling a current limiting component intermediate a elongated conductive member and a source of energy;
shunting excess electrical energy received from an external source.

16. A method according to claim 15, wherein the external source comprises an external defibrillator.

17. A method according to claim 16, wherein the external defibrillator comprises an automated external defibrillator (AED).

18. A method according to claim 15, wherein the voltage-limiting component comprises a varistor.

19. A method according to claim 18, wherein the varistor comprises a metal oxide varistor.

20. An apparatus adapted to shunt electrical energy from an implantable medical device (IMD) when the IMD is subjected to a high voltage defibrillation therapy delivered externally to a patient, comprising:
means for operatively coupling a voltage-limiting component intermediate a elongated conductive member and a source of energy for active circuitry;
means for shunting excess electrical energy received from an external source away from the active circuitry.
